(19) United States
(12) Patent Application Publication
(10) Pub. No.: US 2026/0260711 A1
(43) Pub. Date: Sep. 3, 2026

(54) NON-TRANSITORY COMPUTER-READABLE RECORDING MEDIUM, MOLECULAR ENERGY CALCULATION APPARATUS, AND MOLECULAR ENERGY CALCULATION METHOD

(71) Applicant: Fujitsu Limited, Kawasaki-shi (JP)

(72) Inventor: Satoshi IMAMURA, Kawasaki (JP)

(73) Assignee: Fujitsu Limited, Kawasaki-shi (JP)

(21) Appl. No.: 19/543,787

(22) Filed: Feb. 18, 2026

(30) Foreign Application Priority Data
Feb. 28, 2025 (JP) 2025-032450

(51) Int. Cl. G16C 20/20; G06N 10/60; G16C 10/00

(52) U.S. Cl. CPC G16C 20/20; G06N 10/60; G16C 10/00

(57) ABSTRACT

A non-transitory computer-readable recording medium stores therein a program that causes a computer to execute a process including dividing a molecule into a plurality of fragments, calculating, for each of the fragments, a first eigenvalue for each of bath orbitals included in the fragments, calculating a second value for each of the bath orbitals based on the first eigenvalue, sorting the second values in a descending order and deriving a cumulative distribution function of the second values, determining the number of the bath orbitals for which a cumulative probability of the cumulative distribution function is greater than a predetermined threshold value, selecting the bath orbitals corresponding to the second values from a largest one among the second values sorted in the descending order, in the number equal to the determined number of the bath orbitals, and calculating an energy of the molecule using the selected bath orbitals.

### CROSS-REFERENCE TO RELATED APPLICATION

This application is based upon and claims the benefit of priority of the prior Japanese Patent Application No. 2025-032450, filed on February 28, 2025, the entire contents of which are incorporated herein by reference.

### FIELD

The embodiment discussed herein is related to a molecular energy calculation technology.

### BACKGROUND

By calculating the energy of a molecule, its properties can be identified. For example, the ground-state energy of a molecule can be used to identify the state in which the molecular structure is stable, and the excitation energy can be used to identify the state in which the molecular structure is unstable. Since understanding such molecular properties is useful for drug discovery and the development of novel materials, quantum chemical calculations are highly important.

Patent Literature 1: International Publication Pamphlet No. WO 2022/097298

Patent Literature 2: Japanese Laid-open Patent Publication No. 2024-158044

Patent Literature 3: U.S. Patent Publication No. 2021/0406421

Patent Literature 4: U.S. Patent Publication No. 2023/0359929

### SUMMARY

According to an aspect of an embodiment, a non-transitory computer-readable recording medium stores therein a program that causes a computer to execute a process including dividing a molecule into a plurality of fragments, calculating, for each of the fragments, a first eigenvalue for each of bath orbitals included in the fragments, calculating a second value for each of the bath orbitals based on the first eigenvalue, sorting the second values in a descending order and deriving a cumulative distribution function of the second values, determining the number of the bath orbitals for which a cumulative probability of the cumulative distribution function is greater than a predetermined threshold value, selecting the bath orbitals corresponding to the second values from a largest one among the second values sorted in the descending order, in the number equal to the determined number of the bath orbitals, and calculating an energy of the molecule using the selected bath orbitals.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating the accuracy and computational complexity of molecular energy calculations;

FIG. 2 is a diagram illustrating an example of a fragmentation method;

FIG. 3 is a diagram illustrating issues with energy calculations using the fragmentation method;

FIG. 4 is a diagram illustrating the relationship between the accuracy of molecular energy calculations and the number of bath orbitals;

FIG. 5 is a diagram illustrating a configuration example of a molecular energy calculation apparatus according to the present embodiment;

FIG. 6 is a diagram illustrating an example of the distribution of min(2 - λ, λ) in DMET;

FIG. 7 is a diagram illustrating an example of a method for determining the number of bath orbitals according to the present embodiment;

FIG. 8 is a flowchart illustrating the flow for a molecular energy calculation process according to the present embodiment;

FIG. 9 is a flowchart illustrating an example of the flow for a bath orbital number determination process according to the present embodiment;

FIG. 10 is a diagram illustrating an example of the effects according to the present embodiment; and

FIG. 11 is a diagram illustrating an example of the hardware configuration of the molecular energy calculation apparatus according to the present embodiment.

### DESCRIPTION OF EMBODIMENT

However, the accuracy and computational complexity of molecular energy calculations depend on the basis set and algorithm used. For the calculation with respect to large-scale systems, high-accuracy molecular energy calculations are currently considered to be difficult because the computational complexity increases exponentially.

Preferred embodiments will be explained with reference to accompanying drawings. The present embodiment is not limited to these examples. The examples can be appropriately combined within a range that does not result in any contradictions.

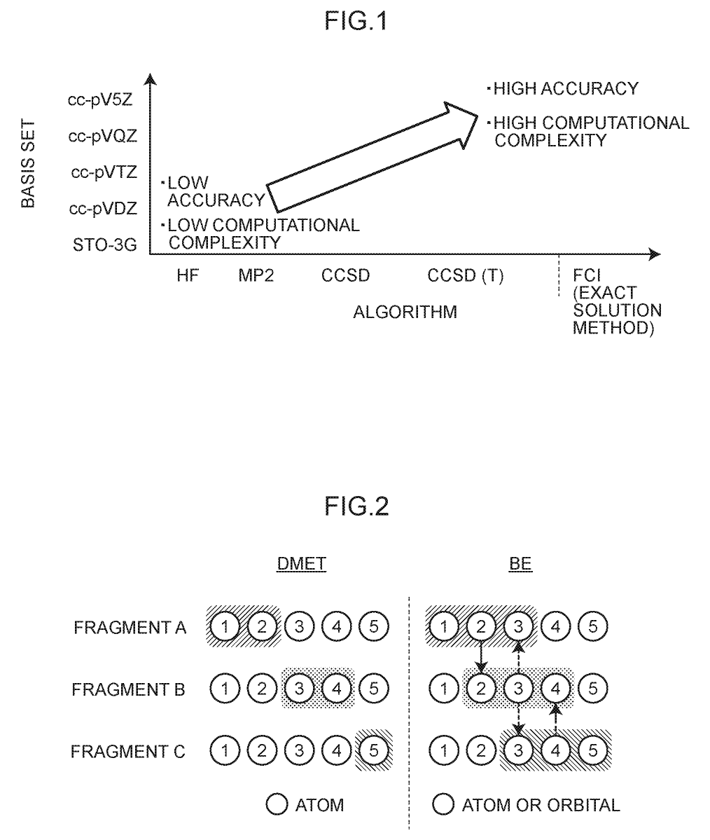

FIG. 1 is a diagram illustrating the accuracy and computational complexity of molecular energy calculations. For the algorithm illustrated in FIG. 1, HF stands for the Hartree Fock method, MP2 stands for the Second-order Moller-Plesset method, and CCSD stands for the Coupling Cluster Singles and Doubles method. CCSD(T) stands for the Coupling Cluster Singles, Doubles, and perturbative Triples method, and FCI stands for the Full Configuration Interaction method.

Each of the basis sets and algorithms used in molecular energy calculations has different accuracy and computational complexity, and is used according to the purposes or other factors. However, for the calculation with respect to large-scale systems, large-scale molecular energy calculations while maintaining high accuracy are currently considered to be difficult because the computational complexity increases exponentially.

Therefore, existing methods, such as DMET and BE, in which a molecule is divided into multiple fragments, the energy of each fragment is calculated, and the total molecular energy is determined by summing the individual fragment energies, have been proposed. Here, the existing method DMET stands for the Density Matrix Embedding Theory, and BE stands for the Bootstrap Embedding. Regarding BE, the present embodiment focuses on the Atom-Based Bootstrap Embedding (ABE), which is BE in atomic units. By using such methods to divide a molecule into multiple fragments, the number of orbitals in the fragments can be reduced as compared to that of the entire molecule, resulting in reduction of the computational complexity.

FIG. 2 is a diagram illustrating an example of a fragmentation method. In FIG. 2, the left side illustrates an example of DMET and the right side illustrates an example of BE. As illustrated in FIG. 2, in DMET, fragments are produced in such a way that atoms do not overlap with one another. On the other hand, in BE, fragments are produced by incrementally offsetting atoms (or orbitals) in such a way that each fragment differs from the previous one by a single atom.

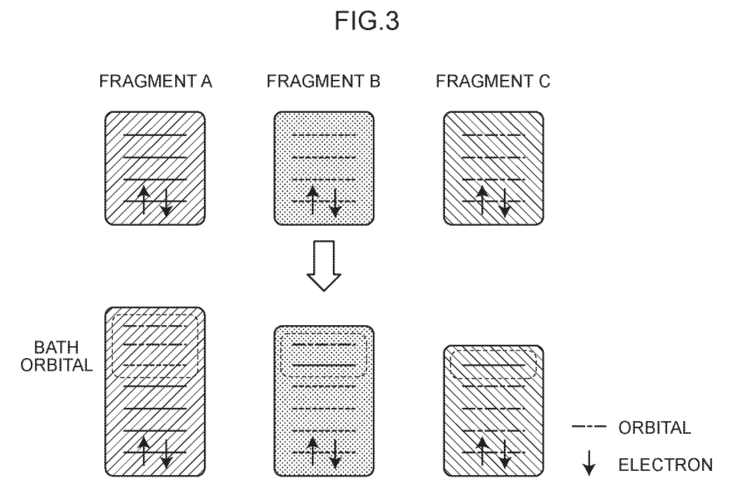

FIG. 3 is a diagram illustrating issues with energy calculations using the fragmentation method. As illustrated in FIG. 3, each fragment that has been divided by a method such as DMET or BE includes, for example, orbitals belonging to the fragment itself and a bath orbital that is a partial orbital from another fragment. In the example in FIG. 3, the orbitals illustrated within the dashed lines are bath orbitals. In this way, since a bath orbital belongs to each fragment, the surrounding environment not included in the fragments, for example, the interaction between fragments, can be represented, which enables an improvement in the accuracy of the calculation.

However, the number of bath orbitals illustrated in FIG. 3 is merely an example, and the number varies depending on fragments. Furthermore, the number of bath orbitals with the highest accuracy varies depending on molecules. On the other hand, the greater the number of bath orbitals, the greater the computational complexity. Therefore, failure to appropriately set the number of bath orbitals may lead to issues such as reduced accuracy in molecular energy calculations and increased computational complexity.

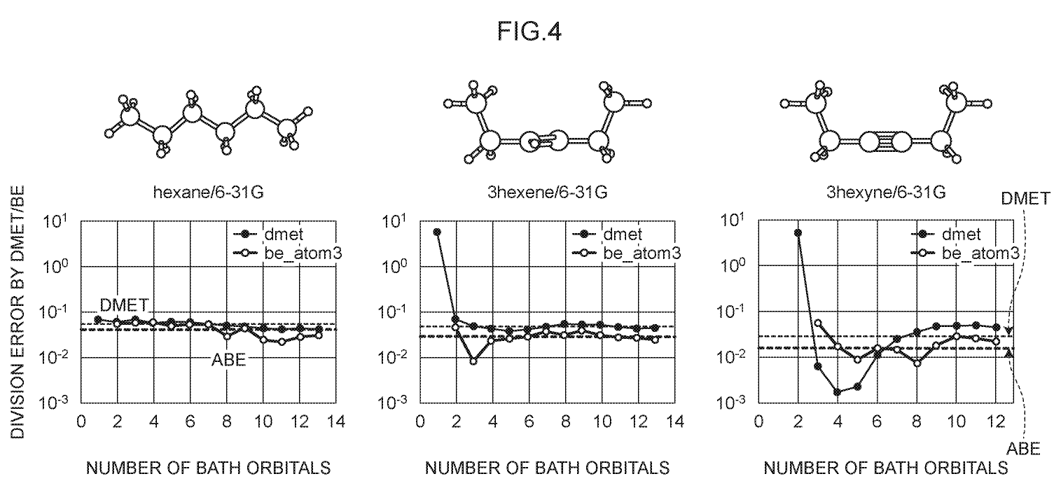

FIG. 4 is a diagram illustrating the relationship between the accuracy of molecular energy calculations and the number of bath orbitals. FIG. 4 illustrates the relationship between the number of bath orbitals and the calculation accuracy thereof when energy calculations on three types of molecules, including hexane/6-31G, are performed. In FIG. 4, the calculation accuracy for each number of bath orbitals can be evaluated by comparison with the error (hereinafter, referred to as "division error") with respect to the total molecular energy obtained with a high-accuracy algorithm (for example, the CCSD method) without using DMET or BE. In FIG. 4, the horizontal dotted lines indicate the division errors in a case where all bath orbitals are included. The phrase "all bath orbitals" refers to all bath orbitals remaining after excluding the bath orbitals determined to be less likely to affect energy calculations by a threshold value against the function "min(2 - λ, λ - 0)" described later. For the reduction in the number of bath orbitals, it is desirable to achieve an error that is equal to or less than that obtained in a case where all bath orbitals are included.

For example, referring to the molecule hexane/6-31G illustrated on the leftmost side in FIG. 4, it can be seen that in the calculation using ABE, the error is smaller and the accuracy is higher in the cases where the numbers of bath orbitals are 8, 10, 11, and the like, as compared to the calculation including all bath orbitals as indicated by the horizontal dotted line. Similarly, in the examples of the molecule 3-hexene/6-31G illustrated in the center in FIG. 4 and the molecule 3-hexyne/6-31G illustrated on the rightmost side in FIG. 4, there are instances where high accuracy is achieved even with a small number of bath orbitals, as compared to the calculation including all bath orbitals. In contrast, in some cases, the accuracy may significantly decrease depending on the number of bath orbitals. As described above, since the appropriate number of bath orbitals varies depending on factors such as the molecules, the algorithms used, and even among fragments, it is really difficult to determine the number of bath orbitals.

Accordingly, an object of the present embodiment is to reduce computational complexity while maintaining accuracy, by determining the minimum number of bath orbitals capable of achieving accuracy equal to or greater than that obtained using all bath orbitals, and calculating molecular energy using only the determined minimum number of bath orbitals.

Functional Configuration of Molecular Energy Calculating Apparatus 10

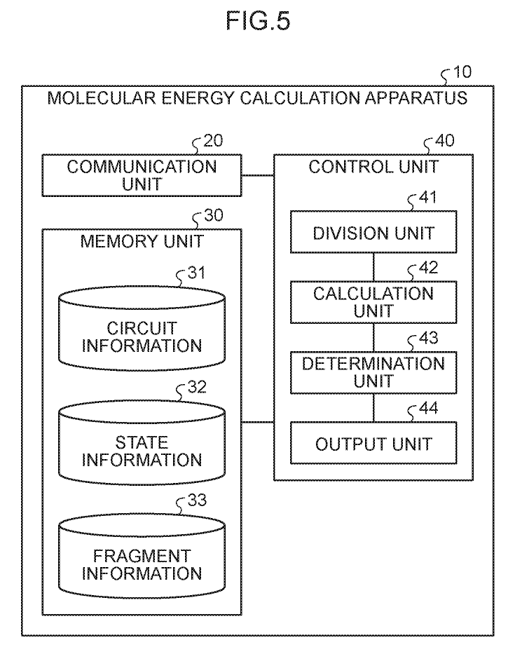

Next, the functional configuration of a molecular energy calculation apparatus 10 serving as the main operating element in the present embodiment will be described. FIG. 5 is a diagram illustrating a configuration example of the molecular energy calculation apparatus according to the present embodiment. The molecular energy calculation apparatus 10 illustrated in FIG. 5 is, for example, a quantum computer such as a noisy intermediate-scale quantum computer (NISQ). The molecular energy calculation apparatus 10 may also be an information processing apparatus such as a server computer or a desktop personal computer (PC), for example. Although the molecular energy calculation apparatus 10 is illustrated as a single computer in FIG. 5, it may instead be configured as a distributed computing system including a plurality of computers. For example, a part or the entirety of the molecular energy calculation apparatus 10 may be a cloud computing apparatus managed by a service provider offering cloud computing services.

As illustrated in FIG. 5, the molecular energy calculation apparatus 10 includes, for example, a communication unit 20, a memory unit 30, and a control unit 40.

The communication unit 20 includes a processing unit that controls communication with other information processing apparatuses and the like, and for example, that is a communication interface such as a network interface card or a universal serial bus (USB) interface.

The memory unit 30 has the function of storing various data and computer programs executed by the control unit 40, and is implemented as, for example, a memory, hard disk, or other storage device. The memory unit 30 stores circuit information 31, state information 32, and fragment information 33.

The circuit information 31 stores, for example, information on a circuit executed by the molecular energy calculation apparatus 10. The information on such a circuit is, for example, information on a digital circuit for executing algorithms such as CCSD. Alternatively, the information on the circuit may be, for example, information on a parameterized quantum circuit for executing a quantum algorithm such as the variational quantum eigensolver (VQE) that is a variational algorithm for NISQ. In quantum chemical calculations, VQE is an algorithm that can search for ground states and calculate ground energies using quantum states.

The state information 32 stores, for example, information and the like on the state of bits input when the molecular energy calculation apparatus 10 executes a digital circuit. The state information 32 may store, for example, information and the like on the state of bits output from digital circuits executed by the molecular energy calculation apparatus 10. Alternatively, information on the quantum state, which is the state of the qubit input when the molecular energy calculation apparatus 10 executes a quantum circuit, may be stored. The state information 32 may store, for example, information and the like on the quantum state output from a quantum circuit executed by the molecular energy calculation apparatus 10.

The fragment information 33 stores, for example, information and the like on fragments divided using DMET or ABE. More specifically, the fragment information 33 may store, for example, the orbitals and bath orbitals of each fragment, the number of orbitals for each fragment, and the energy and the number of electrons calculated for each fragment.

The above information stored in the memory unit 30 is merely an example, and the memory unit 30 may store various types of information other than the above-described information.

The control unit 40 is a processing unit that controls the entire molecular energy calculation apparatus 10, and for example, is a processor. The control unit 40 includes a division unit 41, a calculation unit 42, a determination unit 43, and an output unit 44. Each processing unit is an example of an electronic circuit that is included in the processor or a process that is executed by the processor.

The division unit 41 divides a molecule into a plurality of fragments using, for example, DMET, ABE, or the like.

For example, the calculation unit 42 calculates, for each fragment divided by the division unit 41, a first eigenvalue for each of bath orbitals included in the fragment. Here, the first eigenvalue is, for example, a numerical value between the numerical values 0 and 2, and is represented as an eigenvalue λ (0 ≤ λ ≤ 2), or the like. For example, λ = 0 indicates that a bath orbital is a virtual orbital not occupied by an electron, while λ = 2 indicates that a bath orbital is an occupied orbital occupied by two electrons. As λ is closer to the numerical value of 1, the orbital is likely to affect the Hamiltonian calculation. In contrast, in a case where λ is far from 1, for example, the virtual orbital with λ = 0 and the occupied orbital with λ = 2, such bath orbitals are less likely to affect the Hamiltonian calculation. Accordingly, the accuracy is less likely to decrease when the bath orbitals are excluded from the calculation target. Therefore, by excluding such bath orbitals less likely to affect the accuracy from the calculation target, the computational complexity can be reduced while minimizing a decrease in accuracy in the molecular energy calculation.

A calculation method of the eigenvalue λ, which is the first eigenvalue, is described in more details below. The calculation unit 42 calculates the Hamiltonian of the entire molecule using, for example, an existing low-accuracy method such as the hartree-fock (HF) method, and generates a one-electron density matrix representing the electronic state of the entire molecule. Next, the calculation unit 42 extracts the submatrix corresponding to a bath orbital from the one-electron density matrix for each fragment, for example. Then, the calculation unit 42, for example, diagonalizes the extracted submatrix and calculates the first eigenvalue for each fragment.

The calculation unit 42 also calculates a second value for each of the bath orbitals based on the first eigenvalue, for example. Here, the second value indicates, for example, closeness to the numerical value of 1 of the eigenvalue λ, which is the first eigenvalue, and can be calculated using the function "min(2 - λ, λ)". For example, the function "min(2 - λ, λ)" outputs 1 when λ = 1 is input and outputs 0 when λ = 0 or 2 is input. That is, by using the function "min(2 - λ, λ)", it can be determined that the closer the output value is to 1, the closer the eigenvalue λ of a bath orbital is to 1, whereas the closer the output value is to 0, the farther the eigenvalue λ of a bath orbital is from 1. The function "min(2 - λ, λ)" is strictly a function "min(2 - λ, λ - 0)".

As described above, the bath orbital having the eigenvalue λ that indicates a value of λ farther from the numerical value of 1 is less likely to affect the Hamiltonian calculation. Therefore, for example, by setting a predetermined threshold value for the function "min(2 - λ, λ - 0)" and excluding bath orbitals having values smaller than the threshold value from the calculation target, it is possible to reduce the computational complexity while minimizing a decrease in accuracy in the molecular energy calculation. The threshold value for the function "min(2 - λ, λ - 0)" is, for example, 1E - 13(10⁻¹³), but may be smaller than or greater than this value.

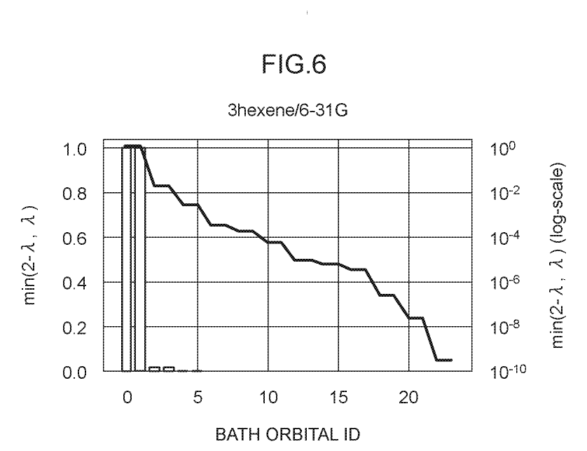

FIG. 6 is a diagram illustrating an example of the distribution of min(2 - λ, λ) in DMET. FIG. 6 illustrates the output values obtained by calculating an eigenvalue λ for each bath orbital included in a fragment divided using DMET and inputting the eigenvalue into the function "min(2 - λ, λ - 0)" in descending order.

In FIG. 6, the left vertical axis and the bar graph represent the output values obtained by the function "min(2 - λ, λ - 0)" on a linear scale, and the right vertical axis and the line graph represent the output values obtained by the function "min(2 - λ, λ - 0)" on a log scale. In the example in FIG. 6, bath orbitals having output value smaller than the threshold value "1E - 13" are excluded.

Referring to FIG. 6, it can be seen that only a limited number of bath orbitals indicated by bath orbitals with IDs 0 to 5 have the output values obtained by the function "min(2 - λ, λ - 0)" that are close to 1. Here, although bath orbitals with IDs 2 to 5 are observed to have output values close to 0 when viewed on the linear scale, the bath orbitals with IDs 2 to 5 are close to 1 when viewed on the log scale, which indicates that only a limited number of bath orbitals affect the Hamiltonian calculation relative to the total number of bath orbitals. In contrast, in FIG. 6, bath orbitals whose output values obtained by the function "min(2 - λ, λ - 0)" are nearly 0, that is, bath orbitals from ID 6 onward, are considered to hardly affect the Hamiltonian calculation.

Therefore, for example, in the example in FIG. 6, the threshold value for the function "min(2 - λ, λ - 0)" can be set to 1E-3(10⁻³) or the like to exclude the bath orbitals from ID 6 onward. Therefore, it is possible to reduce the computational complexity while minimizing a decrease in accuracy by excluding the bath orbitals from ID 6 onward, which are less likely to affect the Hamiltonian calculation, and performing molecular energy calculations using the remaining six bath orbitals, that is, the bath orbitals with IDs 0 to 5.

However, it is not easy to set an appropriate value for the threshold value for the function "min(2 - λ, λ - 0)" because, for example, the threshold value varies depending on molecules. Therefore, in the present embodiment, the cumulative distribution function (CDF) of the output values obtained by the function "min(2 - λ, λ - 0)" is used to determine the number of bath orbitals that affect the Hamiltonian calculation, that is, the number of bath orbitals used in the molecular energy calculation.

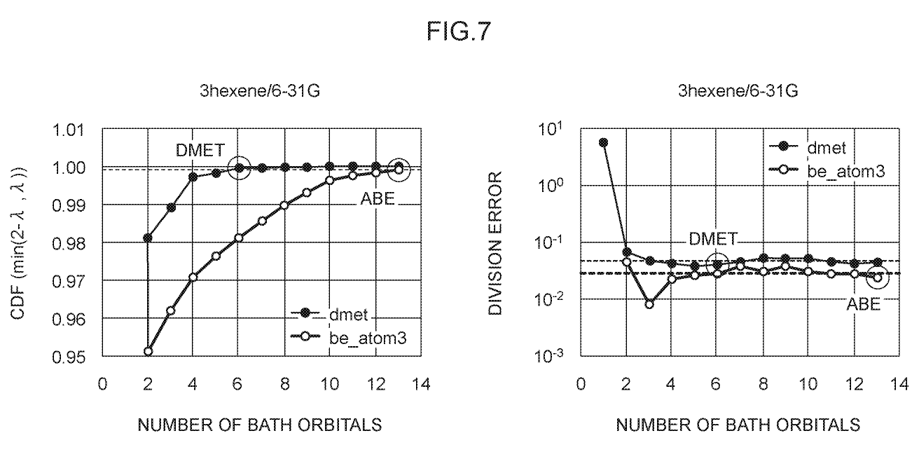

Accordingly, the calculation unit 42, for example, sorts the calculated second values in descending order to derive the cumulative distribution function of the second values. FIG. 7 is a diagram illustrating an example of a method for determining the number of bath orbitals according to the present embodiment. The graph on the left side in FIG. 7 illustrates the output values obtained by sorting the values of min(2 - λ, λ - 0) in descending order for bath orbitals included in fragments of the molecule 3-hexene/6-31G, which have been divided using DMET and ABE, and inputting the sorted values into the cumulative distribution function.

Next, for example, in the example illustrated on the left side in FIG. 7, a threshold value such as 0.999 is set for the cumulative probability, and the minimum number of bath orbitals greater than the threshold value is determined as the number of bath orbitals that affect the Hamiltonian calculation. The threshold value is indicated by a horizontal dotted line in the example on the left side in FIG. 7. In the example on the left side of FIG. 7, the circled bath orbital numbers 6 and 13 in the plots for each of DMET and ABE are determined as the number of bath orbitals that affect the Hamiltonian calculation. That is, the determination unit 43 illustrated in FIG. 5 determines, for example, the number of bath orbitals for which a cumulative probability of the cumulative distribution function derived by the calculation unit 42 is greater than a predetermined threshold value.

On the other hand, the graph on the right side in FIG. 7 is the graph in the center in FIG. 4, which illustrates the relationship between the accuracy of the energy calculation for the molecule 3-hexene/6-31G, and the number of bath orbitals, and in which the number of bath orbitals determined to have a cumulative probability greater than a predetermined threshold value is indicated with circles. Referring to the example on the right side in FIG. 7, it can be seen that, in both cases of DMET and ABE, the determined number of bath orbitals achieves accuracy equal to or higher than that obtained when all bath orbitals are used in the calculation.

Then, the determination unit 43 selects, for example, the bath orbitals corresponding to the second values from the largest one among the second values sorted in descending order, in a number equal to the number of bath orbitals determined to have a cumulative probability greater than a predetermined threshold value. Here, the second values are, for example, the output values obtained by the function "min(2 - λ, λ - 0)" for the bath orbitals calculated by the calculation unit 42. In the example on the left side in FIG. 7, the number of bath orbitals for which a cumulative probability of the cumulative distribution function is greater than a predetermined threshold value is determined to be six in a case where DMET is used for the molecule 3-hexene/6-31G, as described above. Therefore, as illustrated in FIG. 6, the six bath orbitals, that is, the bath orbitals with IDs 0 to 5, are selected from the largest one among the output values sorted in descending order, and obtained by the function "min(2 - λ, λ - 0)" for each bath orbital of the molecule 3-hexene/6-31G.

The calculation unit 42 calculates, for example, the energy of the molecule using the bath orbitals selected by the determination unit 43. As a result, the molecular energy calculation can be performed using only a minimum number of bath orbitals, thereby reducing the computational complexity while maintaining the calculation accuracy. The calculation unit 42 can also calculate the energy of the molecule using, for example, a second method for quantum chemical calculation more accurate than the first method for quantum chemical calculation. Here, the first method is, for example, an existing low-accuracy method such as the hartree-fock (HF) method. On the other hand, the second method is, for example, VQE, CCSD, or the like.

Returning to the description of FIG. 5, the output unit 44 outputs, for example, the molecular energy calculated by the calculation unit 42. Here, the output of the molecular energy may be, for example, a visual display on a display of the molecular energy calculation apparatus 10, or transmission to another information processing apparatus communicably connected to the molecular energy calculation apparatus 10.

### Flow of Processing

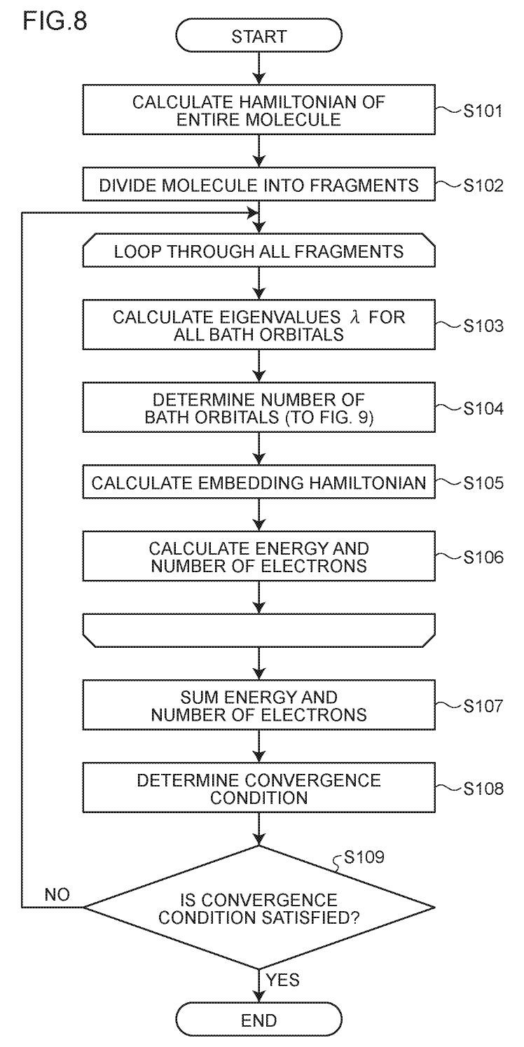

Next, FIG. 8 is used to explain a molecular energy calculation process performed by the molecular energy calculation apparatus 10. FIG. 8 is a flowchart illustrating the flow for the molecular energy calculation process according to the present embodiment.

First, as illustrated in FIG. 8, the molecular energy calculation apparatus 10 generates a Hamiltonian for the entire molecule by calculating the Hamiltonian using, for example, an existing low-accuracy method such as the Hartree Fock (HF) method (step S101).

Next, the molecular energy calculation apparatus 10 divides the molecule into a plurality of fragments using, for example, DMET, ABE, or the like (step S102). The processes at subsequent steps S103 to S106 are repeatedly executed for each of the fragments divided at step S102, looping through all the fragments.

Next, the molecular energy calculation apparatus 10 calculates eigenvalues λ, each of which is a first eigenvalue, for all bath orbitals included in the fragments divided at step S102, for example (step S103). More specifically, the molecular energy calculation apparatus 10 generates, for example, a one-electron density matrix representing the electronic state of the entire molecule using the Hamiltonian of the entire molecule generated at step S101. Next, the molecular energy calculation apparatus 10 extracts, for example, a submatrix corresponding to the bath orbitals from the generated one-electron density matrix and calculates the eigenvalue λ for each of the bath orbitals by diagonalizing the extracted submatrix.

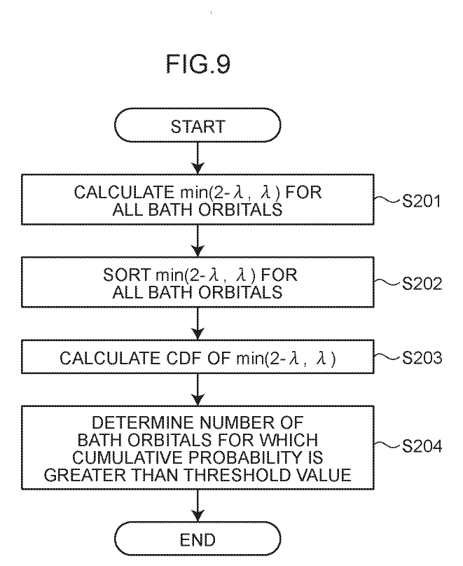

Next, the molecular energy calculation apparatus 10 determines the number of bath orbitals to be used for molecular energy, for example (step S104). An example of a bath orbital number determination process at step S104 will be described more specifically using FIG. 9.

FIG. 9 is a flowchart illustrating an example of the flow for the bath orbital number determination process according to the present embodiment. First, as illustrated in FIG. 9, the molecular energy calculation apparatus 10 obtains an output value by calculating the function "min(2 - λ, λ - 0)" using each eigenvalue λ corresponding to each of the bath orbitals calculated at step S103 as an input value (step S201).

Next, the molecular energy calculation apparatus 10 sorts all the bath orbitals in descending order based on the output values obtained by the function "min(2 - λ, λ - 0)" for all the bath orbitals calculated at step S201 (step S202).

Next, the molecular energy calculation apparatus 10 calculates a cumulative distribution function (CDF) by inputting the output values obtained by the function "min(2 - λ, λ - 0)" for all the bath orbitals sorted in descending order at step S202, thereby obtaining the result (step S203).

Next, the molecular energy calculation apparatus 10 determines the number of bath orbitals for which a cumulative probability is greater than a predetermined threshold value, such as 0.999, using the calculation result of the cumulative distribution function obtained at step S203 (step S204). After the process at step S204 is executed, the bath orbital number determination process illustrated in FIG. 9 ends, and the process returns to the molecular energy calculation process at step S104 illustrated in FIG. 8.

Next, the molecular energy calculation apparatus 10 calculates and generates an embedding Hamiltonian that includes bath orbitals in a number equal to the number of bath orbitals determined at step S204 (step S105). Specifically, the molecular energy calculation apparatus 10 selects the bath orbitals corresponding to the output values from the largest one among the output values sorted in descending order and obtained by the function "min(2 - λ, λ - 0)" for all the bath orbitals, in a number equal to the number of bath orbitals determined at step S204. The molecular energy calculation apparatus 10 then calculates and generates an embedding Hamiltonian that includes the selected bath orbitals. The molecular energy calculation apparatus 10 then calculates the embedding Hamiltonian using an existing method such as CCSD or VQE, and calculates a density matrix representing an electronic state.

Next, the molecular energy calculation apparatus 10 calculates the energy of each fragment based on the density matrix calculated at step S105 (step S106). The molecular energy calculation apparatus 10 also calculates the number of electrons included in a fragment including bath orbitals (step S106). Then, in a case where the processes of steps S103 to S106 have been executed for all the fragments divided at step S102, the molecular energy calculation apparatus 10 proceeds to step S107.

Next, the molecular energy calculation apparatus 10 sums the energy and the number of electrons of the fragments calculated at step S106 (step S107).

Next, the molecular energy calculation apparatus 10 determines whether a predetermined convergence condition is satisfied (step S108 and step S109). Here, the predetermined convergence condition is, for example, a condition as to whether or not the number of electrons summed at step S107 matches the number of electrons of the entire molecule. In a case where the predetermined convergence condition is not satisfied, such as both electron numbers do not match (No at step S109), the molecular energy calculation apparatus 10 updates a penalty value for the Hamiltonian, and restarts the processing from step S103.

On the other hand, in a case where both electron numbers match (Yes at step S109), the molecular energy calculation apparatus 10 outputs the energy summed at step S107 as the molecular energy, and ends the molecular energy calculation process illustrated in FIG. 8.

### Effects

As described above, the molecular energy calculation apparatus 10 divides a molecule into a plurality of fragments, calculates, for each of the fragments, a first eigenvalue for each of bath orbitals included in the fragments, calculates a second value for each of the bath orbitals based on the first eigenvalue, derives a cumulative distribution function of the second values by sorting the second values in a descending order, determines the number of the bath orbitals for which a cumulative probability of the cumulative distribution function is greater than a predetermined threshold value, selects the bath orbitals corresponding to the second values from the largest one among the second values sorted in the descending order, in a number equal to the determined number of the bath orbitals, and calculates an energy of the molecule using the selected bath orbitals.

As described above, the molecular energy calculation apparatus 10 derives a cumulative distribution function by sorting, in descending order, values for respective bath orbitals based on eigenvalues of the bath orbitals included in fragments obtained by dividing a molecule, and calculates molecular energy using the bath orbitals for which a cumulative probability is greater than a threshold value. Accordingly, the molecular energy calculation apparatus 10 can calculate molecular energy using only a minimum number of bath orbitals, which enable calculation accuracy to be retained in molecular energy calculations, resulting in reduction of the computational complexity.

The process of dividing the molecule into the fragments executed by the molecular energy calculation apparatus 10 may include a process of dividing a molecule into a plurality of fragments using DMET or ABE.

Accordingly, the molecular energy calculation apparatus 10 can reduce the computational complexity in molecular energy calculations using DMET or ABE.

The process of calculating the second value executed by the molecular energy calculation apparatus 10 includes a process of calculating, based on the first eigenvalue, the second value indicating closeness to the numerical value of 1, which is one of eigenvalues for the bath orbitals.

Accordingly, since the molecular energy calculation apparatus 10 can calculate molecular energy while excluding bath orbitals that are less likely to affect the Hamiltonian calculation, it is possible to reduce the computational complexity.

The process of calculating the first eigenvalue, which is executed by the molecular energy calculation apparatus 10, includes a process of calculating the first eigenvalue for each fragment using the first method for quantum chemical calculation, and the process of calculating the energy of the molecule includes a process of calculating the energy of the molecule using the second method for quantum chemical calculation more accurate than the first method for quantum chemical calculation, and the selected bath orbitals.

Accordingly, the molecular energy calculation apparatus 10 can selectively use methods for quantum chemical calculation according to the desired calculation accuracy, resulting in reduction of the computational complexity of molecular energy calculation.

The process of calculating the first eigenvalue, which is executed by the molecular energy calculation apparatus 10, includes a process of calculating, for each fragment, the first eigenvalue that is a numerical value between the numerical value of 0 and the numerical value of 2, and the process of calculating the second value indicating closeness to the numerical value of 1 includes a process of calculating the second value indicating closeness to the numerical value of 1 using the first eigenvalue as λ, and the function min(2 - λ, λ).

Accordingly, since the molecular energy calculation apparatus 10 can calculate molecular energy while excluding bath orbitals that are less likely to affect the Hamiltonian calculation, it is possible to reduce the computational complexity.

The molecular energy calculation apparatus 10 generates a one-electron density matrix representing the electronic state of the entire molecule, and the process of calculating the first eigenvalue, which is performed by the molecular energy calculation apparatus 10, includes a process of extracting, for each fragment, a submatrix corresponding to the bath orbitals from the one-electron density matrix, diagonalizing the extracted submatrix, diagonalizing the extracted submatrix and calculating the first eigenvalue for each fragment.

Accordingly, the molecular energy calculation apparatus 10 can reduce the computational complexity in molecular energy calculations.

In order to further demonstrate the effects of the present embodiment, it is presented as comparative data indicating that the accuracy and execution time between a case where molecular energy calculation is performed using all bath orbitals and a case where molecular energy calculation is performed using a limited number of bath orbitals according to the present embodiment.

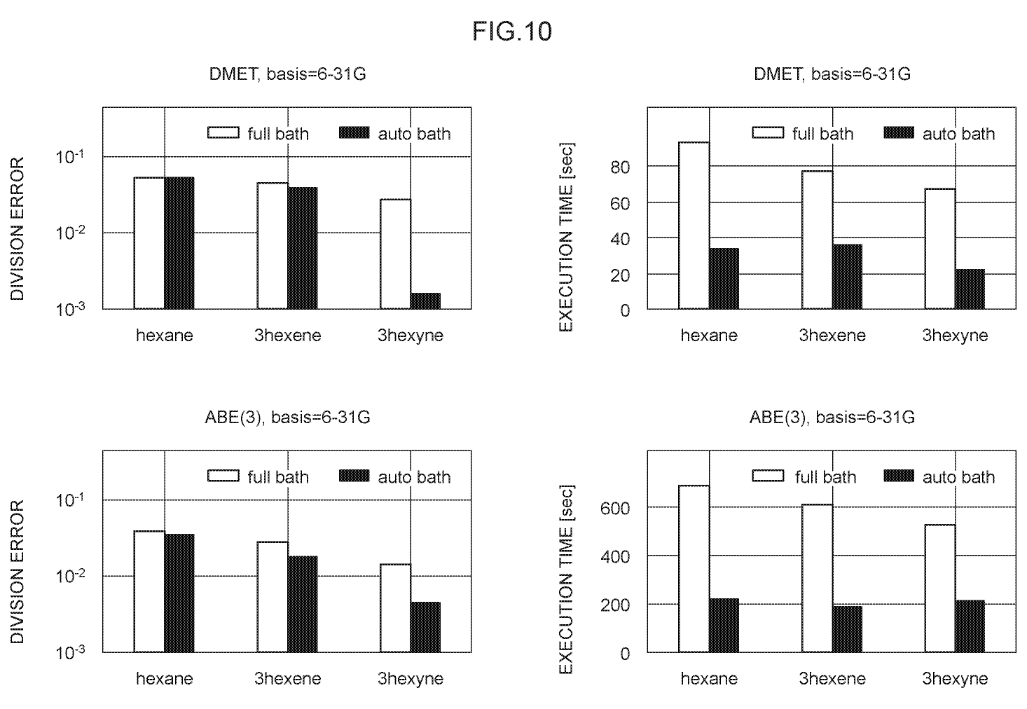

FIG. 10 is a diagram illustrating an example of the effects according to the present embodiment. The left two graphs in FIG. 10 are graphs illustrating the accuracy of each of molecular energy calculations performed for three types of molecules, such as hexane/6-31G, and each of the two graphs on the rightmost side is a graph illustrating the execution time. In FIG. 10, "full bath" refers to data obtained by performing molecular energy calculation using all bath orbitals, and "auto bath" refers to data obtained by performing molecular energy calculation using a limited number of bath orbitals according to the present embodiment. The upper two graphs in FIG. 10 are graphs in a case where DMET is used, and the lower two graphs are graphs in a case where ABE is used. In FIG. 10, "(3)" of "ABE (3)" indicates the number 3 representing the number of non-hydrogen atoms included in each of the fragments divided by ABE.

As illustrated in FIG. 10, in the case of molecular energy calculation using a limited number of bath orbitals according to the present embodiment, the execution time is significantly reduced as compared to the case using all bath orbitals, while achieving equal or higher accuracy regardless of whether DMET or ABE is used.

### System

Unless otherwise specified, the processing procedures, control procedures, specific names, and various pieces of information including data and parameters represented in the above-described text and drawings may be changed as desired. The specific examples, distributions, numerical values, and the like described in the examples are only examples and may be changed as desired.

The specific forms of distribution or integration of the components of the molecular energy calculation apparatus 10 are not limited to the illustrated examples. For example, the calculation unit 42 of the molecular energy calculation apparatus 10 may be distributed across a plurality of the processing units, or the calculation unit 42 and the determination unit 43 of the molecular energy calculation apparatus 10 may be integrated into a single processing unit. That is, all or some of the components may be functionally or physically distributed or integrated in any units, depending on various factors such as processing load or usage conditions. Furthermore, all or some of the individual processing functions of the individual apparatuses can be implemented by a central processing unit (CPU) and a computer program analyzed and executed by the CPU, or may be implemented as hardware by wired logic.

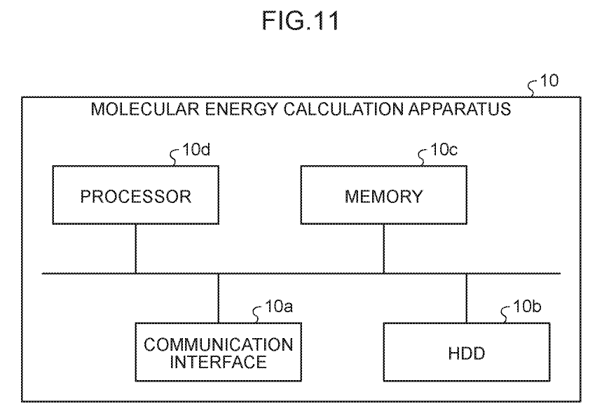

FIG. 11 is a diagram illustrating an example of the hardware configuration of the molecular energy calculation apparatus according to the present embodiment. As illustrated in FIG. 11, the molecular energy calculation apparatus 10 includes a communication interface 10a, a hard disk drive (HDD) 10b, a memory 10c, and a processor 10d. The individual components illustrated in FIG. 11 are interconnected via a bus or the like.

The communication interface 10a is a network interface card or the like, and communicates with other information processing apparatuses. The HDD 10b stores, for example, computer programs and data that operate functions of the molecular energy calculation apparatus 10.

The processor 10d is a CPU, a micro processing unit (MPU), a graphics processing unit (GPU), or the like. The processor 10d may be implemented by an integrated circuit such as an application specific integrated circuit (ASIC), a field programmable gate array (FPGA), or the like. The processor 10d reads out, for example, computer programs for executing processes similar to those of the processing units illustrated in FIG. 5 and the like, from the HDD 10b or the like, and loads the computer programs into the memory 10c. Accordingly, the processor 10d can operate as a hardware circuit that executes processes for implementing various functions of the molecular energy calculation apparatus 10.

The molecular energy calculation apparatus 10 reads out, for example, computer programs for executing processes similar to those of the processing units illustrated in FIG. 5 and the like, from a recording medium via a medium reader. Then, the molecular energy calculation apparatus 10 can implement, for example, functions similar to those of the above-described examples by executing the computer programs thus read out. The computer programs are not limited to being executed by the molecular energy calculation apparatus 10. For example, the computer programs may be executed by another information processing apparatus, or may be executed collaboratively by the other information processing apparatus and the molecular energy calculation apparatus 10.

The computer programs may be distributed via a network such as the Internet. The computer programs may be recorded on a computer-readable storage medium such as a hard disk, a flexible disk (FD), a CD-ROM, a magneto-optical disk (MO), or a digital versatile disc (DVD). Then, the computer programs may be executed by being read out from the recording medium by the molecular energy calculation apparatus 10 or the like.

In one aspect, the computational complexity of molecular energy calculation can be reduced.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention has(have) been described in detail, it should be understood that the various changes, substitutions, and alterations could be made hereto without departing from the spirit and scope of the invention.

What is claimed is:

1. A non-transitory computer-readable recording medium having stored therein a program that causes a computer to execute a process comprising:
dividing a molecule into a plurality of fragments;
calculating, for each of the fragments, a first eigenvalue for each of bath orbitals included in the fragments;
calculating a second value for each of the bath orbitals based on the first eigenvalue;
sorting the second values in a descending order and deriving a cumulative distribution function of the second values;
determining the number of the bath orbitals for which a cumulative probability of the cumulative distribution function is greater than a predetermined threshold value;
selecting the bath orbitals corresponding to the second values from a largest one among the second values sorted in the descending order, in the number equal to the determined number of the bath orbitals; and
calculating an energy of the molecule using the selected bath orbitals.

2. The non-transitory computer-readable recording medium according to claim 1, wherein the dividing into the fragments includes dividing the molecule into the fragments by using density matrix embedding theory (DMET) or atom-based bootstrap embedding (ABE).

3. The non-transitory computer-readable recording medium according to claim 1, wherein the calculating of the second value includes calculating, based on the first eigenvalue, the second value indicating closeness to a numerical value of 1 that is one of eigenvalues for the bath orbitals.

4. The non-transitory computer-readable recording medium according to claim 3, wherein
the calculating of the first eigenvalue includes calculating, for each fragment, the first eigenvalue that is a numerical value between a numerical value of 0 and a numerical value of 2, and
the calculating of the second value indicating closeness to the numerical value of 1 includes calculating the second value indicating closeness to the numerical value of 1 using the first eigenvalue as λ, and a function min(2 - λ, λ).

5. The non-transitory computer-readable recording medium according to claim 1, wherein the process further includes calculating a Hamiltonian of an entirety of the molecule using a first method for quantum chemical calculation and generating a one-electron density matrix representing an electronic state of the entirety of the molecule, wherein
the calculating of the first eigenvalue includes
extracting, for each fragment, a submatrix corresponding to the bath orbitals from the one-electron density matrix, and
calculating the first eigenvalue for each fragment by diagonalizing the extracted submatrix.

6. The non-transitory computer-readable recording medium according to claim 5, wherein the calculating of the energy of the molecule includes calculating the energy of the molecule using a second method for quantum chemical calculation more accurate than the first method, and the selected bath orbitals.

7. A molecular energy calculation apparatus comprising:
a processor configured to:
divide a molecule into a plurality of fragments;
calculate, for each of the fragments, a first eigenvalue for each of bath orbitals included in the fragments;
calculate a second value for each of the bath orbitals based on the first eigenvalue;
sort the second values in a descending order and derive a cumulative distribution function of the second values;
determine the number of the bath orbitals for which a cumulative probability of the cumulative distribution function is greater than a predetermined threshold value;
select the bath orbitals corresponding to the second values from a largest one among the second values sorted in the descending order, in the number equal to the determined number of the bath orbitals; and
calculating an energy of the molecule using the selected bath orbitals.

8. The molecular energy calculation apparatus according to claim 7, wherein the processor is configured to divide the molecule into the fragments by using density matrix embedding theory (DMET) or atom-based bootstrap embedding (ABE).

9. The molecular energy calculation apparatus according to claim 7, wherein the processor is configured to calculate, based on the first eigenvalue, the second value indicating closeness to a numerical value of 1 that is one of eigenvalues for the bath orbitals.

10. The molecular energy calculation apparatus according to claim 9, wherein the processor is configured to:
calculate, for each fragment, the first eigenvalue that is a numerical value between a numerical value of 0 and a numerical value of 2, and
calculate the second value indicating closeness to the numerical value of 1 using the first eigenvalue as λ, and a function min(2 - λ, λ).

11. The molecular energy calculation apparatus according to claim 7, wherein the processor is further configured to calculate a Hamiltonian of an entirety of the molecule using a first method for quantum chemical calculation and generating a one-electron density matrix representing an electronic state of the entirety of the molecule, wherein the processor is configured to:
extracte, for each fragment, a submatrix corresponding to the bath orbitals from the one-electron density matrix, and
calculate the first eigenvalue for each fragment by diagonalizing the extracted submatrix.

12. The molecular energy calculation apparatus according to claim 11, wherein the processor is configured to calculate the energy of the molecule using a second method for the quantum chemical calculation more accurate than the first method, and the selected bath orbitals.

13. A molecular energy calculation method comprising:
dividing a molecule into a plurality of fragments;
calculating, for each of the fragments, a first eigenvalue for each of bath orbitals included in the fragments;
calculating a second value for each of the bath orbitals based on the first eigenvalue;
sorting the second values in a descending order and deriving a cumulative distribution function of the second values;
determining the number of the bath orbitals for which a cumulative probability of the cumulative distribution function is greater than a predetermined threshold value;
selecting the bath orbitals corresponding to the second values from a largest one among the second values sorted in the descending order, in the number equal to the determined number of the bath orbitals; and
calculating an energy of the molecule using the selected bath orbitals, using a processor.

14. The molecular energy calculation method according to claim 13, wherein the dividing into the fragments includes dividing the molecule into the fragments by using density matrix embedding theory (DMET) or atom-based bootstrap embedding (ABE).

15. The molecular energy calculation method according to claim 13, wherein the calculating of the second value includes calculating, based on the first eigenvalue, the second value indicating closeness to a numerical value of 1 that is one of eigenvalues for the bath orbitals.

16. The molecular energy calculation method according to claim 15, wherein
the calculating of the first eigenvalue includes calculating, for each fragment, the first eigenvalue that is a numerical value between a numerical value of 0 and a numerical value of 2, and
the calculating of the second value indicating closeness to the numerical value 1 includes calculating the second value indicating closeness to the numerical value 1 using the first eigenvalue as λ, and a function min(2 - λ, λ).

17. The molecular energy calculation method according to claim 13, wherein the process further includes calculating a Hamiltonian of an entirety of the molecule using a first method for quantum chemical calculation, and generating a one-electron density matrix representing an electronic state of the entirety of the molecule, wherein
the calculating of the first eigenvalue includes
extracting, for each fragment, a submatrix corresponding to the bath orbitals from the one-electron density matrix, and
calculating the first eigenvalue for each fragment by diagonalizing the extracted submatrix.

18. The molecular energy calculation method according to claim 17, wherein the calculating of the energy of the molecule includes calculating the energy of the molecule using a second method for the quantum chemical calculation more accurate than the first method, and the selected bath orbitals.
